(19) United States
(12) Patent Application Publication
(10) Pub. No.: US 2015/0126734 A1
(43) Pub. Date: May 7, 2015

(54) PROCESS FOR PREPARATION OF N,N-DI SUBSTITUTED CARBOXAMIDES

(71) Applicants: Ambati Narasimha Rao, Gwalior (IN); Kumaran Ganesan, Gwalior (IN); Rajagopalan Vijayaraghavan, Gwalior (IN)

(72) Inventors: Ambati Narasimha Rao, Gwalior (IN); Kumaran Ganesan, Gwalior (IN); Rajagopalan Vijayaraghavan, Gwalior (IN)

(73) Assignee: DIRECTOR GENERAL, DEFENCE RESEARCH & DEVELOPMENT ORGNISATION, New Delhi (IN)

(21) Appl. No.: 14/356,001

(22) PCT Filed: Dec. 20, 2011
(86) PCT No.: PCT/IN2011/000873

§ 371 Date: Sep. 12, 2014

(30) Foreign Application Priority Data
Nov. 4, 2011 (IN) 3142/DEL/2011

(51) Int. Cl. C07C 231/14; C07D 213/82; C07D 295/195

(52) U.S. Cl. CPC C07C 231/14; C07D 295/195; C07D 213/82
USPC 544/169; 564/183; 564/215; 546/317

(57) ABSTRACT

The present disclosure relates to a single pot process for preparation of a N,N-di substituted carboxamide compounds of formula (I), said process comprising: reacting a carboxylic acid with a di-substituted carbamoyl chloride in presence of an organic tertiary base to obtain the N,N-di substituted carboxamide compounds of formula (I).

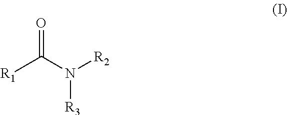

The process of the present disclosure involves a simple step, and it is energy and time saving process for preparation of N,N-di substituted carboxamides.

### TECHNICAL FIELD

The present disclosure relates to a single pot process for preparation of N,N-di substituted carboxamides. The process of the present disclosure is an energy and time saving process.

### BACKGROUND

Amides or carboxamides are important commercial, biological compounds, pharmaceuticals as well as agrochemicals. Amides are used widely in the colouring agents, in crayons, pencils and inks, paper industry, plastic and rubber industry, and water and sewage treatment. Acetaminophen, which is an amide is used as analgesic (pain-killer). It is used as active ingredient in products such as Amadil, Datril, Cetadol, Naprinol, Tylenol, and Panadol. Another example of amide analgesic is phenacetin, used in products such as Empirin and APC (aspirin, phenacetin, and caffeine) tablets. Other commercially used amides includes DEB, DEPA, DEET which are used as insect repellents; lidocaine (Xylocaine) and dibucaine (Nupercaine), used as the local anaesthetics; the tranquilizer meprobromate (Equaine, Miltown); and Sevin and Mipcin used as insecticides.

The various general methods for the preparation of carboxamides are disclosed in literatures.

The classical method disclosed in Vogel's Text book of Practical Organic Chemistry 5^{th} Edition, Longman, New York, 1989, page no. 708-710 and J. March, Advanced Organic Chemistry, John Wiley & Sons, New York, 1992, page no. 416-425, involves conversion of the carboxylic acid into its acid chloride by reaction with thionyl chloride followed by the addition of amine in an anhydrous organic solvent at low temperature to give corresponding carboxamide.

Indian patent application No: 199/DEL/2008 discloses a process for preparation of N,N-Diethyl-2-phenylacetamide (DEPA) comprising reaction of phenyl acetic acid with excess thionyl chloride at 100° C. The excess thionyl chloride is removed by distillation, and then the phenyl acetyl chloride is treated with diethyl amine in diethyl ether medium at 0-10° C. The desired product, DEPA is extracted with dichloromethane from the water soluble by-product, diethylamide hydrochloride. Pure DEPA is obtained by vacuum distillation. This method can be used for the bulk production of DEPA industrially.

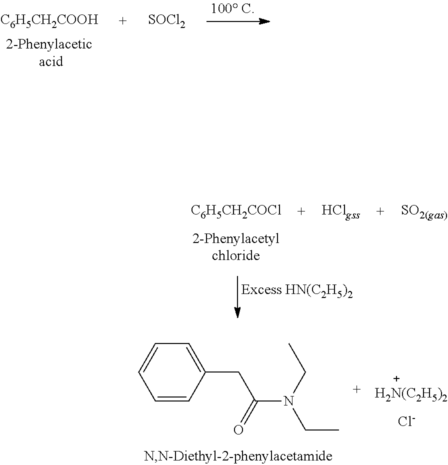

The disadvantages of this process are:
(i) Thionyl chloride is a moisture sensitive reagent, hence difficult to handle large quantities.
(ii) The by-products of thionyl chloride reaction are acidic gases namely hydrogen chloride and sulphurdioxide leading to environmental pollution, responsible for acid rain.
(iii) The thionyl chloride reaction is carried out 80-100° C.
(iv) The diethylamine addition reaction is highly exothermic, hence the reaction is carried out at low temperature, 0-10° C. Organic solvent, diethyl ether is required to control the reaction.
(v) The product obtained from this method is yellow, which is not preferred. The product should be colorless.
(vi) It is a two step process, and requires vacuum distillation in each step to get the product.
(vii) The boiling point of diethylamine is very low (55° C.) thus there is handling and process loss due to evaporation.
(viii) The by-product, solid diethylamine hydrochloride, holds the desired product which leads to low yield of the final product.

Thus this method is more energy utilizing, time consuming and affects environment.

Another process known in the art for preparation of carboxamide involves the reaction of carboxylic acid with triphenylphosphine in carbon tetrachloride or other suitable chloro-compounds to give the acyl chloride, which on reaction with amine gives the desired carboxamide along with triphenylphosphine oxide as by-product. (Ref: Hanan A. Al-hazam., J. Sci. Res. 2009, 1(3), 576-582 & Jang D. O et al., Tetrahedron Lett. 1999, 40, 5323-5326 & L. E. Barstow and V. J. Hruby, J. Org. Chem., 1971, 36, 1305.)

For example, the preparation of the insect repellent, DEB is illustrated below:

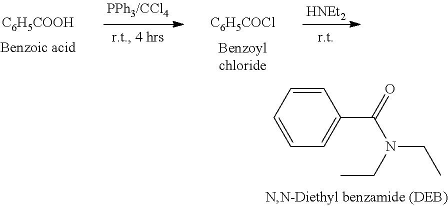

The advantage of preparing acyl chlorides by this method is that the reaction is carried out at room temperature, hence it is energy saving. However, the drawbacks of this method are:
(i) Suitable method to separate the side-product, triphenylphosphine oxide to get the desired product in pure form is yet to be developed.
(ii) The method involves the use of carbon tetrachloride, which is carcinogenic. Further, the supply and use of carbon tetrachloride have been banned due to its effects on the ozone layer in many countries since the year 2002.

Another method for the preparation of carboxamides involves direct reaction of amine with the carboxylic acid in presence of coupling agents or inorganic dehydrating agents. (Ref.: Jaszay Z. M., Petnehazy I. Tock L. Synthesis. 1989, 745-747 European Journal of Scientific Research. 2009, 31, 510-518, & Ali khalafi-nwzhad et al. Tetrahedron Letters. 2005, 46, 6879-6882)

Indian patent No. 166260 discloses the preparation of DEPA in which the reaction of dialkylamine with aryl acetic acid is carried out in presence of inorganic acid as a catalyst at high temperature (100-800° C.) and high pressure (10-800 psi). The drawbacks of this method are:
(i) It requires high temperature which is not suitable for bulk production and hence it is not an energy conservative process.
(ii) The process requires maintenance of high pressure which is difficult and it causes risk from the safety point of view. Since the organic raw materials are flammable, explosion may occur if the method is not performed properly.
(iii) Use of strong inorganic acid (phosphoric acid) is corrosive to the reaction vessel.
(iv) Purification method of the product disclosed is also difficult.

Indian patent No. 169195 provides the preparation of DEPA by the reaction of arylacetic acid with dialkylamine in presence of inorganic acid and organic catalysts. This process suffers from several disadvantages that are as follows:
(i) The maintenance of a high temperature for the reaction to proceed in the forward direction requires high and continuous heating of the apparatus.
(ii) The removal of water formed during the course of the reaction has to be carried out at high temperature; hence it poses a fire hazard and requires special fire safety measures.
(iii) The process is exothermic and forms thick slurry of solids that hinder the stirring of the reaction.
(iv) The process is energy consuming and expensive, as energy is constantly required to maintain the reflux conditions.
(v) The quantity of environmentally hazardous effluent from the process is very high.
(vi) It is applicable to laboratory scale but not suitable for industrial scale.
(vii)

In-situ activation of carboxylic acids by coupling reagents such as N,N-dicyclohexylcarbidimide (DCC), TiC₄, activated phosphate, Sn[N(TMS)₂]₂, N-halosyuccinimnid/Ph₃P, ArB(OH₂)₂, Lowesson's reagent, (R₂N)₂Mg, SO₂CIF, chlorosulfonyl isocyante, and 2-mercaptopyridine-1-oxide based uranium salts have also been reported in the literatures, for example Sheehan J C, Hess G P., J. Am. Chem. Soc. 1955, 77, 1067-1068., Wilson J D, Hobbs C F, Wengaten H., J Org. Chem. 1970, 15, 1542-1545., Yasuhara T. Nagaka Y, Tomioka K J., Chem. Soc. Perkin Trans. 2000, 901-2902., Burnell-Curty C, Roskamp E J., Tetrahedron Lett. 1993, 34, 5193-5196., Froyen P., Synth. Commun. 1995, 25, 959-968., Ishihara K, Ohara S, Yamamoto H., J. Org. Chem, 1996, 61, 4196-4199., Throse J D. Andersen T P, Pedesen U, Yde B, Laweson S., Tetrahedron. 1985, 41, 5633-5636., Sanchez R. Vest G. Depress L., Synth. Commun. 1989, 19, 2909-2913., Olah G A, Narang S C, Lina A G., Synthesis. 1980, 8, 661-662., Keshavamurthy K S, Vankar Y D, Dhar D N., Synthesis, 1982, 506-508., & Bailen M A, Chinchilla R, Dodsworth D J, Najera C., Tetrahedron Lett. 2000, 41, 9809-9813. Thus reaction with amine in the presence of these agents gives the desired carboxamides.

The drawbacks of these methods are:

(i) They are suitable for laboratory scale only.

(ii) The separation of the product involves chromatographic techniques.

(iii) Undesired by-products are formed.

Grega et al provides a process for the production of N,N-disubstituted carboxylic amides in U.S. Pat. No. 3,941,783. In this process, the carboxylic acid is reacted with carbamoyl chloride to obtain the N,N-disubstituted carboxylic amides. The drawbacks of this method are:
(i) The reaction mixture is heated to 110-220° C. making the process energy consuming.
(ii) The gaseous by-product, hydrogen chloride is acidic and corrosive in nature. It is released to the atmosphere; otherwise a suitable method has to be used to prevent this in manufacturing industries. Also it will affect the gas linings outgoing from the reaction vessel.
(iii) The carboxamides formed becomes yellowish in colour due to the higher reaction temperature used.
(iv) The method is applicable to limited starting materials as there is a possibility of side reaction at high temperature conditions.
(v) The product yield depends on the completion of the reaction which is determined by the time when the gas evolution from the reaction stops. This is difficult to apply in industrial scale.

Keeping in view the requirement of energy conserving industrial process in the present scenario, there is a need to develop a simple and cost effective method for the preparation and process development of N,N-Di substituted carboxamides which can be upscaled to commercial level which is also devoid of the disadvantages/drawbacks of the processes known in the art.

### SUMMARY

The present disclosure provides a single pot process for preparation of a N,N-di substituted carboxamide compounds of formula (I), said process comprising: reacting a carboxylic acid of formula (II) with a di-substituted carbamoyl chloride of formula (III) in presence of an organic tertiary base for a time period in the range of 15 minutes to 60 minutes, and at a temperature in the range of 10° C. to 50° C. to obtain the N,N-di substituted carboxamide compounds of formula (I)

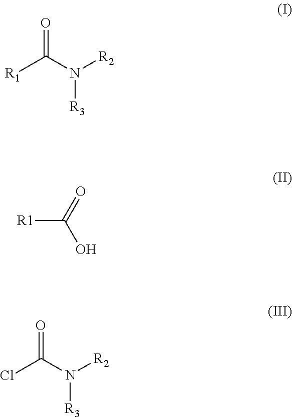

wherein:
R₁, R₂ and R₃ are each independently selected from an optionally substituted alkyl or an optionally substituted aryl.

The present disclosure provides a single pot process for preparation of a N,N-di substituted carboxamide compounds which involves simple step, and is energy and time saving which can also be up-scaled for the commercial manufacturing of N,N-di substituted carboxamide compounds.

These and other features, aspects, and advantages of the present subject matter will become better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### DETAILED DESCRIPTION

The present disclosure provides a single pot process for preparation of a N,N-di substituted carboxamide compounds of formula (I), said process comprising: reacting a carboxylic acid of formula (II) with a di-substituted carbamoyl chloride of formula (III) in presence of an organic tertiary base for a time period in the range of 15 minutes to 60 minutes, and at a temperature in the range of 10° C. to 50° C. to obtain the N,N-di substituted carboxamide compounds of formula (I)

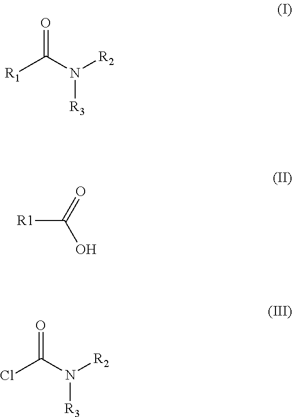

wherein:
R₁, R₂ and R₃ are each independently selected from an optionally substituted alkyl or an optionally substituted aryl.

An embodiment of the present disclosure provides a single pot process for preparation of a N,N-di substituted carboxamide compounds of formula (I), said process comprising: reacting a aromatic carboxylic acid of formula (II) with a di-substituted carbamoyl chloride of formula (III) in presence of an organic tertiary base for a time period in the range of 15 minutes to 60 minutes, and at a temperature in the range of 10° C. to 50° C. to obtain the N,N-di substituted carboxamide compounds of formula (I)

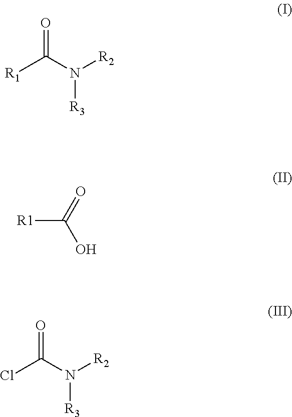

wherein:
R₁ is selected from optionally substituted aryl; and
R₂ and R₃ are each independently selected from an optionally substituted alkyl or an optionally substituted aryl.

Another embodiment of the present disclosure provides a single pot process fir preparation of a N,N-di substituted carboxamide compounds of formula (I), said process comprising: reacting a aliphatic carboxylic acid of formula (II) with a di-substituted carbamoyl chloride of formula (III) in presence of an organic tertiary base for a time period in the range of 15 minutes to 60 minutes, and at a temperature in the range of 10° C. to 50° C. to obtain the N,N-di substituted carboxamide compounds of formula (I)

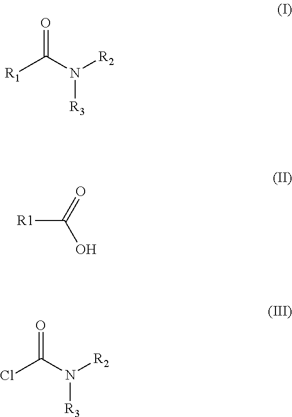

wherein:
R₁ is selected from optionally substituted alkyl; and
R₂ and R₃ are each independently selected from an optionally substituted alkyl or an optionally substituted aryl.

The term “optionally substituted” as used in the disclosure means that the group is either unsubstituted or substituted with one or more substituents. When the group is substituted with more than one substituent, the substituent may be same or different. The substituents in accordance with the present disclosure is selected from halogen, —OH, oxo, cyano, aryl, heteroaryl, hetercyclyl, cycloalkyl, alkyl, alkoxy, —CONH2, or —NH2.

The term “alkyl” referred here includes both branched and straight-chain saturated aliphatic hydrocarbon groups, including all isomers, cycloalkyl, heterocyclyl and the likes. Alkyl in accordance with the present disclosure can have 1 to 10 carbon atoms. Non limiting examples of alkyl groups include methyl, ethyl, propyl, isopropyl, butyl, t-butyl and the like.

The term “cycloalkyl” means mono-, bi- or tri-cyclic structures, optionally combined with linear or branched structures.

The term “heterocyclyl” means a 3- to 10-membered non-aromatic monocyclic or bicyclic ring containing one or more heteroatoms selected from O, S or N.

The term “aryl” is defined as a mono- or bi-cyclic aromatic ring system and includes heteroaryl, aralkyl and the likes. Non limiting examples of aryl include phenyl, biphenyl, naphthyl or anthryl.

The term heteroaryl means 5- to 10-membered aromatic, partially aromatic mono- or bicyclic ring, containing 1-4 heteroatoms selected from O, S or N.

The term “aralkyl” means an alkyl group as defined above of 1 to 6 carbon atoms with an aryl group as defined above substituted for one of the alkyl atoms.

The carboxylic acid of formula (II), in accordance with the present disclosure, is either an aromatic or an aliphatic carboxylic acid. Non limiting examples of carboxylic acid in accordance with the present disclosure are phenyl acetic acid, 3-methyl benzoic acid, benzoic acid, morpholine 4-carboxylic acid, nicotinic acid, propionic acid, butanoic acid, pentanoic acid, octanoic acid, hexadecanoic acid or octadecanoic acid.

Non limiting examples of the di-substituted carbomyl chloride of formula (III) in accordance with the present disclosure are N,N-dimethyl carbamoyl chloride, N,N-diethyl carbomyl chloride, N,N-dibutyl carbomyl chloride, N-methyl-N-ethyl carbomyl chloride, or N-propyl-N-ethyl carbomyl chloride.

Non-limiting examples of the organic tertiary base in accordance with the present disclosure are triethylamine, tributylamine, 1-methyl imidazole, pyridine, piperidine, N-methyl pyrrolidine, N-methyl pyrrole, N-methyl piperidine and 4-methyl morpholine. The use of the organic tertiary base in the present disclosure is an important feature since the organic tertiary base initiates the reaction at room temperature and speeds up the reaction. Further, the organic tertiary base scavenges the HCl gas by product formed during formation of the quaternary salt. Since the quaternary salt formed is soluble in water, it can be easily separated from the final product.

The process of the present disclosure takes not more than 60 minutes for completion. For example, if the tertiary base is triethylamine, the reaction is completed within 15 minutes and if the tertiary base is I-methyl imidazole, the reaction is completed within 30 minutes.

An embodiment of the present disclosure provides a single pot process for preparation of a N,N-di substituted carboxamide compounds of formula (I), said process comprising: reacting a carboxylic acid of formula (II) with a di-substituted carbamoyl chloride of formula (III) in presence of triethylamine for a time period in the range of 15 minutes to 60 minutes, and at a temperature in the range of 10° C. to 50° C. to obtain the N,N-di substituted carboxamide compounds of formula (I).

Another embodiment of the present disclosure provides a single pot process for preparation of a N,N-di substituted carboxamide compounds of formula (I), said process comprising: reacting a carboxylic acid of formula (II) with a di-substituted carbamoyl chloride of formula (III) in presence of tributylamine for a time period in the range of 15 minutes to 60 minutes, and at a temperature in the range of 10° C. to 50° C. to obtain the N,N-di substituted carboxamide compounds of formula (I).

Yet another embodiment of the present disclosure provides a single pot process for preparation of a N,N-di substituted carboxamide compounds of formula (I), said process comprising: reacting a carboxylic acid of formula (II) with a di-substituted carbamoyl chloride of formula (III) in presence of 1-methyl imidazole for a time period in the range of 15 minutes to 60 minutes, and at a temperature in the range of 10° C. to 50° C. to obtain the N,N-di substituted carboxamide compounds of formula (I).

Still, another embodiment of the present disclosure provides a single pot process for preparation of a N,N-di substituted carboxamide compounds of formula (I), said process comprising: reacting a carboxylic acid of formula (II) with a di-substituted carbamoyl chloride of formula (III) in presence of piperidine for a time period in the range of 15 minutes to 60 minutes, and at a temperature in the range of 10° C. to 50° C. to obtain the N,N-di substituted carboxamide compounds of formula (I).

Further, an embodiment of the present disclosure provides a single pot process for preparation of a N,N-di substituted carboxamide compounds of formula (I), said process comprising: reacting a carboxylic acid of formula (II) with a di-substituted carbamoyl chloride of formula (III) in presence of N-methyl pyrrolidine for a time period in the range of 15 minutes to 60 minutes, and at a temperature in the range of 10° C. to 50° C. to obtain the N,N-di substituted carboxamide compounds of formula (I).

Another embodiment of the present disclosure provides a single pot process for preparation of a N,N-di substituted carboxamide compounds of formula (I), said process comprising: reacting a carboxylic acid of formula (II) with a di-substituted carbamoyl chloride of formula (III) in presence of N-methyl pyrrole for a time period in the range of 15 minutes to 60 minutes, and at a temperature in the range of 10° C. to 50° C. to obtain the N,N-di substituted carboxamide compounds of formula (I).

Yet, another embodiment of the present disclosure provides a single pot process for preparation of a N,N-di substituted carboxamide compounds of formula (I), said process comprising: reacting a carboxylic acid of formula (II) with a di-substituted carbamoyl chloride of formula (III) in presence of N-methyl piperidine for a time period in the range of 15 minutes to 60 minutes, and at a temperature in the range of 10° C. to 50° C. to obtain the N,N-di substituted carboxamide compounds of formula (I).

Still, another embodiment of the present disclosure provides a single pot process for preparation of a N,N-di substituted carboxamide compounds of formula (I), said process comprising: reacting a carboxylic acid of formula (II) with a di-substituted carbamoyl chloride of formula (III) in presence of 4-methyl morpholine for a time period in the range of 15 minutes to 60 minutes, and at a temperature in the range of 10° C. to 50° C. to obtain the N,N-di substituted carboxamide compounds of formula (I).

Still, another embodiment of the present disclosure provides a single pot process for preparation of a N,N-di substituted carboxamide compounds of formula (I), said process comprising: reacting a carboxylic acid of formula (II) with a di-substituted carbamoyl chloride of formula (II) in presence of pyridine for a time period in the range of 15 minutes 25 to 60 minutes, and at a temperature in the range of 10° C. to 50° C. to obtain the N,N-di substituted carboxamide compounds of formula (I).

The process of the present disclosure occurs at room temperature. The room temperature for the purposes of the present disclosure may vary from 10° C. to 50° C. preferably 20° C. to 45° C. more preferably 25° C. to 40° C. If the room temperature is low the reaction time is more, whereas if the room temperature is high, the reaction time is low.

An embodiment of the present disclosure provides a single pot process for preparation of a N,N-di substituted carboxamide compounds of formula (I), said process comprising: reacting phenyl acetic acid with N,N-diethyl carbamoyl chloride in presence of 1-methylimidazole for a time period in the range of 25 to 35 minutes, and at a temperature in the range of 10° C. to 50° C. to obtain N,N-diethyl-2-phenyl acetamide.

Yet another embodiment of the present disclosure provides a single pot process for preparation of a N,N-di substituted carboxamide compounds of formula (I), said process comprising: reacting 3-methyl benzoic acid with N,N-diethyl carbamoyl chloride in presence of triethylamine for a time period in the range of 15 to 25 minutes, and at a temperature in the range of 10° C. to 50° C. to obtain N,N-diethyl-m-toulamide.

Still another embodiment of the present disclosure provides a single pot process for preparation of a N,N-di substituted carboxamide compounds of formula (I), said process comprising: reacting benzoic acid with N,N-diethyl carbamoyl chloride in presence of tributylamine for a time period in the range of 15 to 25 minutes, and at a temperature in the range of 10° C. to 50° C. to obtain N,N-diethyl benzamide.

Further, an embodiment of the present disclosure provides a single pot process for preparation of a N,N-di substituted carboxamide compounds of formula (I), said process comprising: reacting octanic acid with N,N-diethyl carbamoyl chloride in presence of triethylamine for a time period in the range of 15 to 25 minutes, and at a temperature in the range of 10° C. to 50° C. to obtain N,N-diethyl octanamide.

Yet another embodiment of the present disclosure provides a single pot process for preparation of a N,N-di substituted carboxamide compounds of formula (I), said process comprising: reacting morpholine-4-carboxylic acid with N,N-dibutyl carbamoyl chloride in presence of pyridine for a time period in the range of 25 to 35 minutes, and at a temperature in the range of 10° C. to 50° C. to obtain N,N-dibutyl morphiline-4-carboxamide.

Still another embodiment of the present disclosure provides a single pot process for preparation of a N,N-di substituted carboxamide compounds of formula (I), said process comprising: reacting nicotinic acid with N,N-dimethyl carbamoyl chloride in presence of I-methyl imidazole for a time period in the range of 25 to 35 minutes, and at a temperature in the range of 10° C. to 50° C. to obtain N,N-dimethyl nicotinamide.

The process of the present disclosure provides a process for preparation of N,N-Di substituted carboxamides; said process comprising: adding 1 mole of aliphatic or aryl carboxylic acid and 1 mole of N,N-di substituted carbamoyl chloride to a 1 liter two-necked round bottom flask equipped with air/water condenser, calcium chloride guard tube and mechanical stirrer; adding slowly 1.2 moles of tertiary organic base, with constant stirring through a pressure-equalizing funnel fitted in the side neck of the round bottom flask to obtain a mixture; stirring the mixture for 15 to 60 minutes at room temperature; adding water to the stirred mixture and desired product, N,N-Di substituted carboxamide is separated out from aqueous layer.

The crude N,N-Di substituted carboxamide obtained by the process of the present disclosure is more than 99% pure and it can be further purified by distillation, if necessary, to get more than 99.5% purity for pharmaceutical use.

Further, in another aspect of the present disclosure, the base-hydrochloride salt formed is separated and neutralized with an acid to obtain the free base, which can then be recycled for further reactions.

### EXAMPLES

The disclosure will now be illustrated with working examples, which is intended to illustrate the working of disclosure and not intended to take restrictively to imply any limitations on the scope of the present disclosure. Other examples are also possible which are within the scope of the present disclosure.

### Example 1

### Preparation of N,N-Diethyl-2-phenyl acetamide (DEPA)

136 g (1 Mole) of phenyl acetic acid and 136 g (=127 ml, 1 Mole) N,N-diethyl carbamoyl chloride are taken in a 1 liter two-necked round-bottom flask fitted with air condenser which is placed over a magnetic stirrer. To this, 98 g (96 ml, 1.2 Mole) of I-methylimidazole, which is an organic tertiary base is added using a pressure-equalizing funnel fitted in the side neck of the round bottom flask at room temperature. After complete addition, the reaction mixture is stirred constantly for 30 minutes at room temperature. The reaction mixture is then treated with 250 ml of water and the two layers are separated. Pure and colourless N,N-diethyl-2-phenylacetamide (DEPA) is obtained by vacuum distillation of organic layer which is the product.

Purity of the compound is analyzed using GC-MS which is more than 99.5%. And yield of hr product is 187 g (98%).

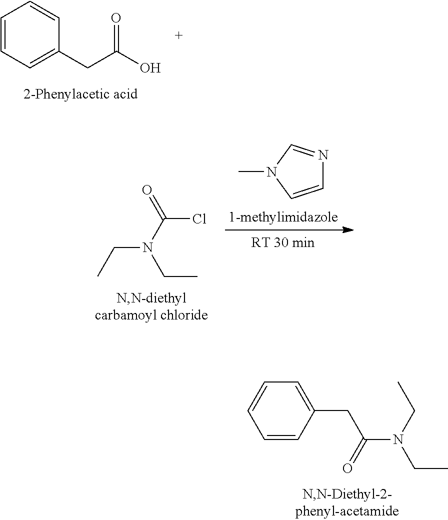

### Example 2

### Preparation of N,N-Diethyl m-Toluamide (DEET)

136 g (1 Mole) of m-toluic acid (3-methyl benzoic acid) and 136 g (=127 ml, 1 Mole) N,N-diethylcarbamoyl chloride are taken in a 1 liter two-necked round-bottom flask fitted with air condenser which is placed over a magnetic stirrer. To this, 121 g (167 ml. 1.2 Mole) of triethylamine, which is a organic base is added using a pressure-equalizing funnel fitted in the side neck of the round bottom flask at room temperature. After complete addition, the reaction mixture is stirred constantly for 20 minutes at room temperature. The reaction mixture is then treated with 250 ml of water and the two layers are separated. Pure and colourless N,N-Diethyl m-toluamide (DEET) is obtained by vacuum distillation of organic layer which is the product.

Purity of the compound is analyzed using GC-MS which is more than 99.5%. The yield of the product is 186 g (97.5%).

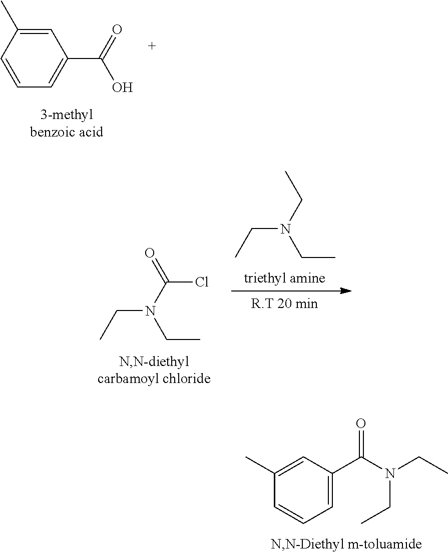

### Example 3

### Preparation of N,N-Diethyl Benzamide (DEB)

122 g (1 Mole) of benzoic acid and 136 g (=127 ml, 1 Mole) N,N-diethylcarbamoyl chloride are taken in a 1 liter two-necked round-bottom flask fitted with air condenser which is placed over a magnetic stirrer. To this, 222 g (285 ml, 1.2 Mole) of tributylamine is added using a pressure-equalizing funnel fitted in the side neck of the round bottom flask at room temperature. After complete addition, the reaction mixture is stirred constantly for 20 minutes at room temperature. The reaction mixture is then treated with 250 ml of water and the two layers are separated. Pure and colorless N,N-diethyl benzamide (DEB) is obtained by vacuum distillation of organic layer which is the product.

Purity of the compound is analyzed using GC-MS, which is more than 99.5%. The yield of the product is 173 g (98%).

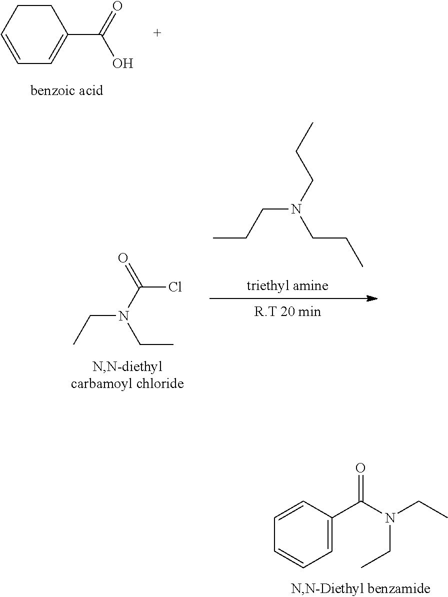

### Example 4

### Preparation of N,N-Diethyl Octanamide

152 g (158 ml, 1 Mol) of octanoic acid and 136 g (127 ml, 1 Mole) N,N-diethyl carbamoyl chloride are taken in a 1 liter two-necked round-bottom flask fitted with air condenser which is placed over a magnetic stirrer. To this, 121 g (167 ml, 1.2 Mole) of triethylamine is added using a pressure-equalizing funnel fitted in the side neck of the round bottom flask at room temperature. After complete addition, the reaction mixture is stirred constantly for 20 minutes at room temperature. The reaction mixture is then treated with 250 ml of water and the two layers are separated. Pure and colorless N,N-diethyl octanamide is obtained by vacuum distillation of organic layer which is the product.

Purity of the compound is analyzed using GC-MS, which is more than 99.5%. The yield of the compound is 195 g (98%).

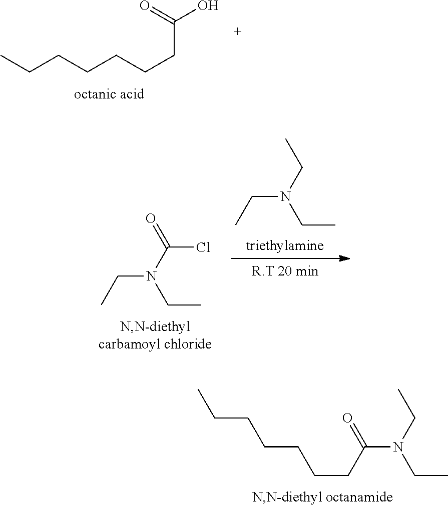

### Example 4

### Preparation of N,N-Dibutyl morpholine-4-carboxamide

131 g (1 Mole) of morpholine-4-carboxylic acid and 191 g (=193 ml, 1 Mole) N,N-dibutylcarbamoyl chloride are taken in a 1 liter two-necked round-bottom flask fitted with air condenser which is placed over a magnetic stirrer. To this, 95 g (97 ml, 1.2 Mole) of pyridine is added using a pressure-equalizing funnel fitted in the side neck of the round bottom flask at room temperature. After complete addition, the reaction mixture is stirred constantly for 30 minutes at room temperature. The reaction mixture is then treated with 250 ml of water and the two layers are separated. Pure and colorless N,N-Dibutyl morpholine-4-carboxamide is obtained by vacuum distillation of organic layer which is the product.

Purity of the compound is analyzed using GC-MS, which is more than >99.5%, the yield of the compound is 237 g (98%).

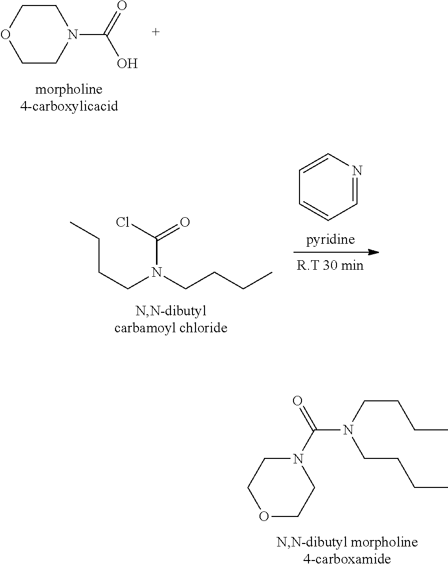 [SMILES: O=C(O)N1CCOCC1 ; c1ccncc1 ; CCCCN(CCCC)C(=O)Cl ; CCCCN(CCCC)C(=O)N1CCOCC1]

### Example 5

### Preparation of N,N-Dimethyl Nicotinamide

123 g (1 Mole) of nicotinic acid and 107 g (=91 ml, 1 Mole) N,N-dimethylcarbamoyl chloride are taken in a 1 liter two-necked round-bottom flask fitted with air condenser which is placed over a magnetic stirrer. To this, 98 g (96 ml. 1.2 Mole) of 1-methylimidazole is added using a pressure-equalizing funnel fitted in the side neck of the round bottom flask at room temperature. After complete addition, the reaction mixture is stirred constantly for 30 minutes at room temperature. The reaction mixture is then treated with 250 ml of water and the two layers are separated. Pure and colorless N,N-Dimethyl nicotinamide is obtained by vacuum distillation of organic layer which is the product.

Purity of the compound is analyzed using GC-MS, which is more than 99.5%. The yield of the compound is 147 g (98%).

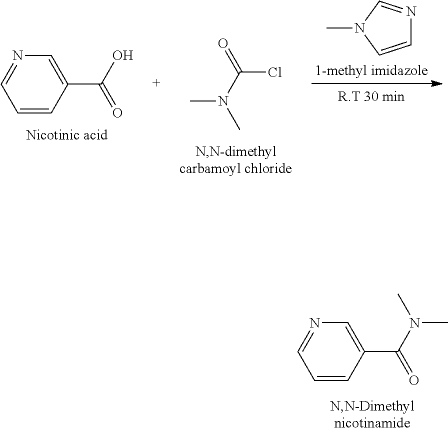

The previously described versions of the subject matter and its equivalent thereof have many advantages, including those which are described below:
The process of the present disclosure is a simple one-pot method for the preparation of N,N-di substituted carboxamide.
The process of the present disclosure is an energy conservative process, as the reaction takes place at room temperature.
The process of the present disclosure does not release any acidic gas in environment and thus the process is environmental friendly.
The process of the present disclosure is a time-saving process.
The process of the present disclosure is cost effective.
The process of the present disclosure can be upscaled easily for bulk production or industrial production.
The yield of the product, N,N-di substituted carboxamide prepared by the present process, is high.
The product, N,N-di substituted carboxamide, obtained from the process of the present disclosure has high purity.
The effluent load in the process of the present disclosure is minimum.

Although the subject matter has been described in considerable detail with reference to certain preferred embodiments thereof, other embodiments are possible. As such, the spirit and scope of the appended claims should not be limited to the description of the preferred embodiment contained therein.

We claim:

1. A single pot process for preparation of a N,N-di substituted carboxamide compounds of formula (I), said process comprising:
reacting a carboxylic acid of formula (II) with a di-substituted carbamoyl chloride of formula (III) in presence of an organic tertiary base for a time period in the range of 15 minutes to 60 minutes, and at a temperature in the range of 10° C. to 50° C. to obtain the N,N-di substituted carboxamide compounds of formula (I) 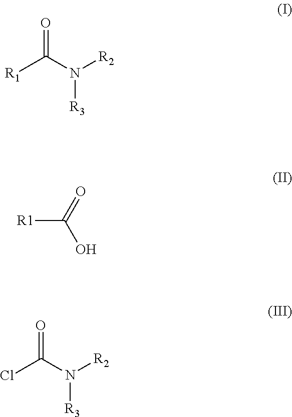
wherein:
R₁, R₂ and R₃ are each independently selected from an optionally substituted alkyl or an optionally substituted aryl.

2. The process as claimed in claim 1, wherein the carboxylic acid is an aromatic or an aliphatic carboxylic acid selected from the group consisting of phenyl acetic acid, 3-methyl benzoic acid, benzoic acid, morpholine 4-carboxylic acid, nicotinic acid, propionic acid, butanoic acid, pentanoic acid, octanoic acid, hexadecanoic acid, or octadecanoic acid.

3. The process as claimed in claim 1, wherein the di-substituted carbomyl chloride is sleeted from the group consisting of N,N-dimethyl carbamoyl chloride, N,N-diethyl carbomyl chloride, N,N-dibutyl carbomyl chloride, N-methyl-N-ethyl carbomyl chloride, and N-propyl-N-ethyl carbomyl chloride.

4. The process as claimed in claim 1, wherein the organic tertiary base is selected from the group consisting of triethylamine, tributylamine, 1-methyl imidazole, pyridine, piperidine, N-methyl pyrrolidine, N-methyl pyrrole, N-methyl piperidine and 4-methyl morpholine.

5. The process a claimed in claim 1, said process comprising:
reacting phenyl acetic acid with N,N-diethyl carbamoyl chloride in presence of 1-methylimidazole for a time period in the range of 25 to 35 minutes, and at a temperature in the range of 10° C. to 50° C. to obtain N,N-diethyl-2-phenyl acetamide.

6. The process a claimed in claim 1, said process comprising:
reacting 3-methyl benzoic acid with N,N-diethyl carbamoyl chloride in presence of triethylamine for a time period in the range of 15 to 25 minutes, and at a temperature in the range of 10° C. to 50° C. to obtain N,N-diethyl-m-toulamide.

7. The process a claimed in claim 1, said process comprising:
reacting benzoic acid with N,N-diethyl carbamoyl chloride in presence of tributylamine for a time period in the range of 15 to 25 minutes, and at a temperature in the range of 10° C. to 50° C. to obtain N,N-diethyl benzamide.

8. The process a claimed in claim 1, said process comprising:
reacting octanic acid with N,N-diethyl carbamoyl chloride in presence of triethylamine for a time period in the range of 15 to 25 minutes, and at a temperature in the range of 10° C. to 50° C. to obtain N,N-diethyl octanamide.

9. The process a claimed in claim 1, said process comprising:
reacting morpholine-4-carboxylic acid with N,N-dibutyl carbamoyl chloride in presence of pyridine for a time period in the range of 25 to 35 minutes, and at a temperature in the range of 10° C. to 50° C. to obtain N,N-dibutyl morphiline-4-carboxamide.

10. The process a claimed in claim 1, said process comprising:
reacting nicotinic acid with N,N-dimethyl carbamoyl chloride in presence of 1-methyl imidazole for a time period in the range of 25 to 35 minutes, and at a temperature in the range of 10° C. to 50° C. to obtain N,N-dimethyl nicotinamide.
